(19) United States
(12) Patent Application Publication
(10) Pub. No.: US 2024/0410109 A1
(43) Pub. Date: Dec. 12, 2024

(54) A METHOD FOR CHEMICAL PULP PRODUCTION IN A MULTI-STAGE PROCESS

(71) Applicant: ANDRITZ AB, Örnsköldsvik (SE)

(72) Inventors: Ola LARSSON, Örnsköldsvik (SE); Michael BERGMANN, Graz (AT); Hannu PYYKÖNEN, Helsinki (FI); Jussi PIIRA, Helsinki (FI); Ari HANNIMÄKI, Lahti (FI); Vesa KAIPAINEN, Kotka (FI); Samuli KINNUNEN, Kotka (FI)

(73) Assignee: ANDRITZ AB, Örnsköldsvik (SE)

(21) Appl. No.: 18/698,077

(22) PCT Filed: Oct. 6, 2022
(86) PCT No.: PCT/SE2022/050899

§ 371 Date: Apr. 3, 2024

(30) Foreign Application Priority Data
Oct. 8, 2021 (FI) 20216040

(51) Int. Cl. D21C 3/22; D21B 1/02; D21B 1/06; D21C 3/26; G01N 33/34

(52) U.S. Cl. CPC D21C 3/228; D21B 1/021; D21B 1/061; D21C 3/26; G01N 33/343

(57) ABSTRACT

A method for chemical pulp production in a multi-stage process, comprising a first process stage, comprising a woodyard comprising steps such as debarking, chipping, pile storage and/or reclaim; a second process stage, comprising a fiberline comprising steps such as cooking and/or bleaching; and at least a third process stage; wherein a first set of data is gathered at least at the first and at the second process stage, wherein the first set of data for each process stage comprises measurement results and/or parameters calculated based on measurement results, wherein the measurements and/or parameters are related to raw material, equipment, process measurements and/or product properties; the first set of data is sent to the subsequent process stage, wherein the process steps of the subsequent process stage are adjusted based on the first set of data received; a second set of data is gathered at least at the second and at the third process stage, wherein the second set of data for each process stage comprises information related to availability of processing capacity, predicted availability of processing capacity and/or required material flow or quality; the second set of data is sent to the preceding process stage, wherein the process steps of the preceding process stage are adjusted based on the second set of data received.

### TECHNICAL FIELD

The present invention relates to a method for chemical pulp production in a multi-stage process. In particular, but not exclusively, the present invention relates to a method for controlling and adjusting chemical pulp production in a multi-stage process.

### BACKGROUND

Pulp production requires stability throughout the manufacturing process so that the quality of the pulp remains stable and the desired quality is reached. In order to provide the end user exactly the required quality, various operational measures are commonly applied.

Previously, the separate stages of pulp manufacturing have been controlled and adjusted in a conventional manner, often each stage or each device separately, by the operators using a control system and interface. Suggestions have been made for control across processing stages, for example publication US2010/0004771 A1 discusses proceeding across different stages according to a predetermined production plan based on need-based optimization. Furthermore, it has been suggested that process data from a manufacturing stage could be delivered to the next stage or to the end user of the product for example by attaching it to a machine readable medium on the product. Such a system has been described in publication WO2014/009605 A1.

It is the object of the present invention to provide a system and method for controlling chemical pulp production throughout the various stages thereof to ensure desired pulp properties and effectiveness of manufacturing.

### SUMMARY

Various aspects of examples of the invention are set out in the claims.

According to a first example aspect of the present invention, there is provided a method for chemical pulp production in a multi-stage process, comprising
a first process stage, comprising a woodyard comprising steps such as debarking, chipping, pile storage and/or reclaim;
a second process stage, comprising a fiberline comprising steps such as cooking and/or bleaching; and
at least a third process stage; wherein
a first set of data is gathered at least at the first and at the second process stage, wherein the first set of data for each process stage comprises measurement results and/or parameters calculated based on measurement results, wherein the measurements and/or parameters are related to raw material, equipment, process measurements and/or product properties;
the first set of data is sent to the subsequent process stage, wherein the process steps of the subsequent process stage are adjusted based on the first set of data received;
a second set of data is gathered at least at the second and at the third process stage, wherein the second set of data for each process stage comprises information related to availability of processing capacity, predicted availability of processing capacity and/or required material flow or quality;
the second set of data is sent to the preceding process stage, wherein the process steps of the preceding process stage are adjusted based on the second set of data received.

The third process stage may comprise a drying line comprising steps such as dewatering, drying and/or baling.

The third process stage may comprise a paper machine, a board machine or a tissue machine.

The first set of data gathered at the first process stage may comprise measurements and/or parameters related to chip quality, wood information, dry mass flow, wood chip storage, log storage and/or key equipment condition.

The first set of data gathered at the second process stage may comprise measurements and/or parameters related to fiber conditions, cooking conditions and/or bleaching conditions.

The second set of data gathered at the second process stage may comprise measurements and/or parameters related to process conditions, desired chip quality, dry mass flow and/or wood species.

The second set of data gathered at the third process stage may comprise measurements and/or parameters related to availability and/or key equipment conditions.

According to a second example aspect of the present invention, there is provided a system for chemical pulp production in a multi-stage process, comprising
a first process stage, comprising a woodyard;
a second process stage, comprising a fiberline; and
at least a third process stage; wherein
at least the first and the second process stage comprise a measurement element and a control element configured to gather a first set of data at least at the first and at the second process stage, wherein the first set of data for each process stage comprises measurement results and/or parameters calculated based on measurement results, wherein the measurements and/or parameters are related to raw material, equipment, process measurements and/or product properties;
the measurement elements and the control elements are configured to send the first set of data to the subsequent process stage, and the measurement element and the control element of the subsequent process stage are configured to adjust the process steps of the subsequent process stage based on the first set of data received;
at least the second and the third process stage comprise a measurement element and a control element configured to gather a second set of data at least at the second and at the third process stage, wherein the second set of data for each process stage comprises information related to availability of processing capacity, predicted availability of processing capacity and/or required material flow or quality; and
the measurement elements and the control elements are configured to send the second set of data to the preceding process stage, and the measurement element and the control element of the preceding process stage are configured to wherein the process steps of the preceding process stage are adjusted based on the second set of data received.

According to a third example aspect of the present invention, there is provided a control system for chemical pulp production in a multi-stage process, comprising a control unit configured to cause carrying out the method according to the first example aspect of the present invention.

According to a fourth example aspect of the present invention, there is provided a computer program product comprising computer-executable code that is configured to, when executed by a computer, to cause carrying out the method according to the first example aspect of the present invention.

According to a fifth example aspect of the present invention, there is provided a non-transitory memory medium comprising the computer program product of the fourth example aspect.

Different non-binding example aspects and embodiments of the present invention have been illustrated in the foregoing. The embodiments in the foregoing are used merely to explain selected aspects or steps that may be utilized in implementations of the present invention. Some embodiments may be presented only with reference to certain example aspects of the invention. It should be appreciated that corresponding embodiments may apply to other example aspects as well.

### BRIEF DESCRIPTION OF THE DRAWINGS

For a more complete understanding of example embodiments of the present invention, reference is now made to the following descriptions taken in connection with the accompanying drawings in which:

FIG. 1 shows a principle flow chart according to an example embodiment of the invention;

FIG. 2 shows a flow chart of a method according to an example embodiment of the invention; and

FIG. 3 shows a schematic block view of a system according to an example embodiment of the invention.

### DETAILED DESCRIPTION OF THE DRAWINGS

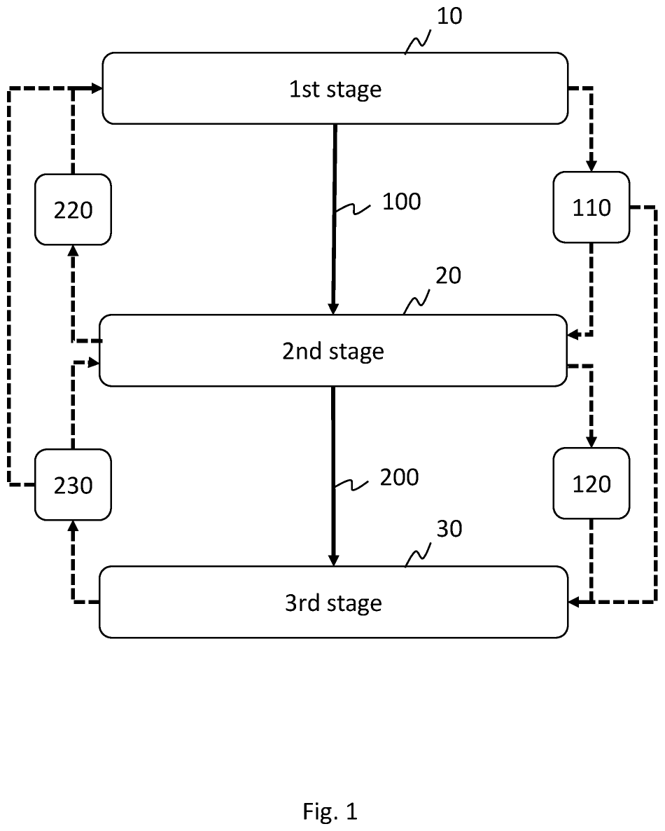

FIG. 1 shows a principle flow chart according to an example embodiment of the invention. The flow chart of FIG. 1 depicts chemical pulp manufacturing having separate process stages 10-30, or separate operational units. In an embodiment, the chemical pulp manufacturing according to an example embodiment of the invention comprises at least a first process stage 10, a second process stage 20 and a third process stage 30. In a further embodiment, the chemical pulp manufacturing according to an example embodiment of the invention comprises further process stages (not shown) to which the embodiments of the invention described hereinafter are equally applicable.

In an embodiment, the first process stage 10 comprises a woodyard and the second process stage 20 comprises a fiberline. In an embodiment, the woodyard process stage comprises steps such as debarking, chipping pile storage and/or reclaim and the fiberline process stage comprises steps such as cooking and/or bleaching. In an example embodiment, the third process stage 30 comprises a drying line, comprising steps such as dewatering, drying and/or baling. In a further example embodiment, the third process stage 20 comprises a paper machine, a board machine or a tissue machine.

FIG. 1 further shows the material and information flows between the process stages 10-30. Material from the first process stage 10, i.e. the end product of the first process stage, flows to the second process stage 20 at 100. Similarly, material from the second process stage 20, i.e. the end product of the second process stage, flows to the third process stage 30 at 200. In an embodiment, the material flow 100 from the first process stage, comprising a woodyard, comprises chips. In an embodiment, the material flow 200 from the second process stage, comprising a fiberline, comprises pulp.

Chemical pulp manufacturing according to example embodiments of the invention is constantly monitored and adjusted. In an embodiment, various data is gathered at all process stages 10-30. According to embodiments of the invention, a first set of data 110,120 is gathered at least at the first 10 and the second 20 process stage. In an embodiment, the first set of data 110,120 for each process stage, respectively, comprises measurement results and/or parameters calculated based on measurement results. In an embodiment, the measurements and/or the parameters are related to raw material, equipment, process measurements and/or product properties as hereinafter explained in further detail.

According to embodiments of the invention, a second set of data 220,230 is gathered at least at the second 20 and the third 30 process stage. In an embodiment, the second set of data 220,230 for each process stage, respectively, comprises information related to availability of processing capacity, predicted availability of processing capacity and/or required material flow or quality as hereinafter explained in further detail.

According to embodiments of the invention, the first set of data 110,120 is sent to the process stage subsequent to the process stage in which it was gathered. Accordingly, the first set of data 110 of the first process stage 10 is sent to the second process stage 20 and the first set of data 120 of the second process stage is sent to the third process stage 30. In a further example embodiment, the first set of data 110 is in addition sent to the process stage subsequent to the subsequent process stage thereof, in an embodiment for adjustment of that stage.

According to embodiments of the invention, the second set of data 220,230 is sent to the process stage preceding the process stage in which it was gathered. Accordingly, the second set of data 220 of the second process stage 20 is sent to the first process stage 10 and the second set of data 230 of the third process stage 30 is sent to the second process stage 20. In a further example embodiment, the second set of data 230 is in addition sent to the process stage preceding the preceding process stage thereof, in an embodiment for adjustment of that stage.

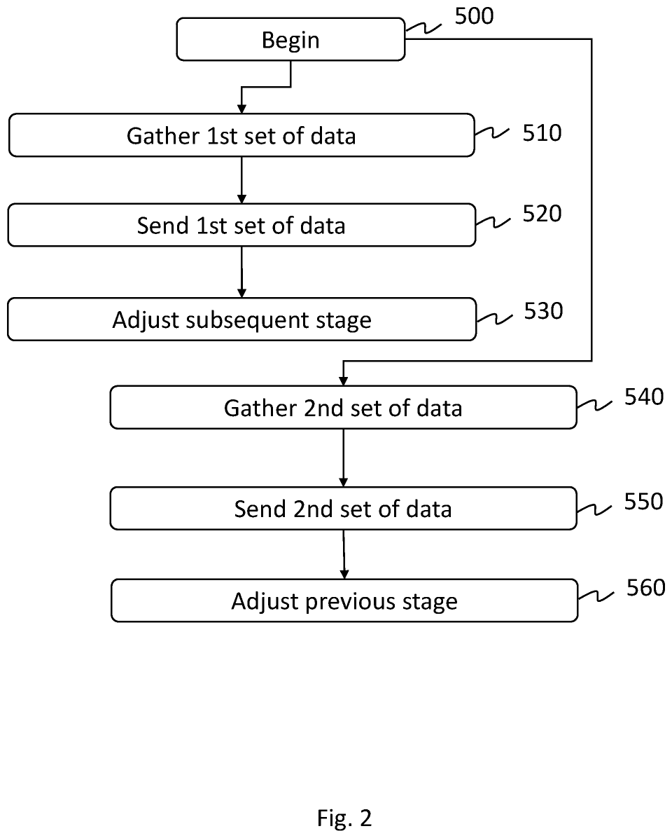

FIG. 2 shows a flow chart of a method according to an example embodiment of the invention. At step 500 the execution of the method is begun. In an embodiment, the method is executed constantly and repeatedly during the production of chemical pulp. In an embodiment, the first 110,120 and the second 220,230 data set are updated constantly and sent in real time. in a further embodiment, the data sets are updated at certain intervals or at predetermined times. While FIG. 2 shows the method according to embodiments of the invention as having two separate branches for ease of understanding, it is to be noted that the operations described are carried out concurrently, i.e. not as separate processes carried out one after the other.

At step 510, the first set of data 110,120 is gathered at least at the first 10 and the second 20 process stage. At step 540, the second set of data 220,230 is gathered ate least at the second 20 and the third 30 process stage.

At step 520 each first set of data 110,120 is sent to the subsequent process stage, respectively. At step 550 each second set of data 220,230 is sent to the preceding process stage, respectively.

In the following, the method according to the invention will be explained in more detail with respect to the example embodiment in which the first process stage 10 comprises a woodyard, the second process stage 20 comprises a fiberline and the third process stage 30 comprises a drying line.

According to the invention, it is recognized that a stable production at high throughput at the fiberline 20 requires an accurate input from the woodyard 20. Accordingly, in an embodiment, the first set of data 110 gathered at the woodyard 10 comprises information selected from but not limited to the measurements and/or parameters shown in Table 1.

TABLE 1

Chip quality
Wood information
Dry mass flow
Wood chip storage information
Log storage information
Key equipment condition

According to the invention, it is further recognized that the woodyard 10 requires input from the fiberline. In an embodiment, the information from the fiberline relates to the current and foreseeable process conditions thereof and in an embodiment comprises requests for chips to be provided that are best fit for the targeted quality and process conditions. In an embodiment, the second set of data 220 gathered at the fiberline comprises information selected from but not limited to the measurements and/or parameters shown in Table 2.

TABLE 2

Process conditions
Stability
Desired chip quality
Dry mass flow
Wood species

It is to be noted, that according to the embodiments of the invention there is provided an awareness between the fiberline 20 and the woodyard 10 about the stock and its conditions in the woodyard, so that also wood chips which exhibit lower quality aspects are processed. According to an embodiment, this is enabled for example by adjusting the output of the woodyard 10 to a mixture with high quality chips at a defined rate or adjusting the production for producing lower quality pulp in case the producer offers different grades to the market.

According to the invention, it is recognized that the stability of the fiberline 20 enables a good runnability or processing availability of the drying line 30 and vice versa. Accordingly, in an embodiment, the first set of data 120 gathered at the fiberline 20 comprises information selected from but not limited to the measurements and/or parameters shown in Table 3.

TABLE 3

Fiber conditions
Cooking conditions
Bleaching conditions

According to the invention, it is further recognized that the fiberline 20 requires input from the drying line 30 in order to enable adapting and optimizing the production of the fiberline. Accordingly, in an embodiment, the second set of data 230 gathered at the drying line 30 comprises information selected from but not limited to the measurements and/or parameters shown in Table 4.

TABLE 4

Availability
Planned stops
Likelihood of interruptions
Key equipment conditions

At steps 530,560 each process stage is adjusted based on the first and/or second set of date received. As an example of the process according to embodiments of the invention, the fiberline process stage receives a first set of data from the woodyard process stage comprising measurement data related to dry mass flow and a digested of the fiberline is adjusted based thereon. As a further example of the process according to embodiments of the invention, the fiberline process stage receives a second set of data from the drying line process stage comprising information related to actual and predicted availability and the production level of the fiberline is adjusted based thereon.

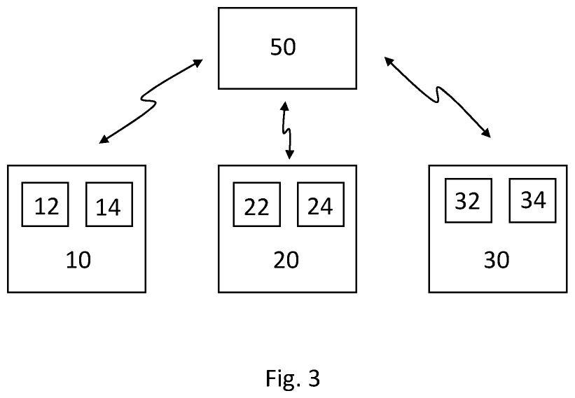

FIG. 3 shows a schematic block view of a system according to an example embodiment of the invention. FIG. 3 shows the first 10, second 20 and the third 30 process stage. Hereto it is noted that the process equipment of each stage is not shown, but it is understood that conventional equipment pertinent to each process stage respectively is used.

Each process stage 10-30 comprises, respectively, a measurement element 12,22,32 and a control element 14,24,34. It is to be noted, that the measurement elements and the control elements contain, in an embodiment several components such as sensors, actuators, electric circuits, hydraulics and processors and are in an embodiment divided into sub-elements, for example in such a way that each piece of processing equipment comprises a dedicated measurement and/or control element. Furthermore, in an embodiment, the measurement and control elements are integrated into a single entity with the desired functionality.

The measurement elements 12,22,32 comprise in an embodiment conventional sensors. In a further embodiment, the measurement elements 12,22,32 comprise soft sensors or virtual sensors in addition to or instead of conventional sensors.

The control elements 14,24,34 comprise in an embodiment conventional actuators. In a further embodiment, the control elements 14,24,34 comprise further units, such as a communication unit, user interface unit and a memory.

The system according to an embodiment of the invention further comprises a control unit 50 in communication with each process stage and the control element 14,24,34 and the measurement element 12,22,32 thereof. In an embodiment, the control unit 50 is comprised in a mill wide control system. In a still further embodiment, the control unit is a virtual control unit, for example a cloud-based control unit. The control unit 50 comprises a processor configured to cause carrying out methods according to embodiments of the invention.

Without in any way limiting the scope, interpretation, or application of the claims appearing below, a technical effect of one or more of the example embodiments disclosed herein is an optimized production process. Another technical effect of one or more of the example embodiments disclosed herein is the provision of improved pulp quality. Another technical effect of one or more of the example embodiments disclosed herein is improved use of raw materials and chemicals. A still further technical effect of one or more of the example embodiments disclosed herein is improved stability of the processes.

If desired, the different functions discussed herein may be performed in a different order and/or concurrently with each other. Furthermore, if desired, one or more of the before-described functions may be optional or may be combined.

Although various aspects of the invention are set out in the independent claims, other aspects of the invention comprise other combinations of features from the described embodiments and/or the dependent claims with the features of the independent claims, and not solely the combinations explicitly set out in the claims.

It is also noted herein that while the foregoing describes example embodiments of the invention, these descriptions should not be viewed in a limiting sense. Rather, there are several variations and modifications which may be made without departing from the scope of the present invention as defined in the appended claims.

1. A method for chemical pulp production in a multi-stage process, comprising:
a first process stage, comprising a woodyard comprising steps such as debarking, chipping, pile storage and/or reclaim;
a second process stage, comprising a fiberline comprising steps such as cooking and/or bleaching; and
at least a third process stage wherein;
a first set of data is gathered at least at the first and at the second process stage, wherein the first set of data for each process stage comprises measurement results and/or parameters calculated based on measurement results, wherein the measurements and/or parameters are related to raw material, equipment, process measurements and/or product properties;
the first set of data is sent to the subsequent process stage, wherein the process steps of the subsequent process stage are adjusted based on the first set of data received;
a second set of data is gathered at least at the second and at the third process stage, wherein the second set of data for each process stage comprises information related to availability of processing capacity, predicted availability of processing capacity and/or required material flow or quality;
the second set of data is sent to the preceding process stage, wherein the process steps of the preceding process stage are adjusted based on the second set of data received.

2. A method according to claim 1, wherein the third process stage comprises a drying line comprising steps such as dewatering, drying and/or baling.

3. A method according to claim 1, wherein the third process stage comprises a paper machine, a board machine or a tissue machine

4. A method according to claim 1, wherein the first set of data gathered at the first process stage comprises measurements and/or parameters related to chip quality, wood information, dry mass flow, wood chip storage, log storage and/or key equipment condition.

5. A method according to claim 1, wherein the first set of data gathered at the second process stage comprises measurements and/or parameters related to fiber conditions, cooking conditions and/or bleaching conditions.

6. A method according to claim 1, wherein the second set of data gathered at the second process stage comprises measurements and/or parameters related to process conditions, desired chip quality, dry mass flow and/or wood species.

7. A method according to claim 1, wherein the second set of data gathered at the third process stage comprises measurements and/or parameters related to availability and/or key equipment conditions.

8. A system for chemical pulp production in a multi-stage process, comprising:
a first process stage, comprising a woodyard;
a second process stage, comprising a fiberline; and
at least a third process stage;
wherein at least the first and the second process stage comprise a measurement element and a control element configured to gather a first set of data at least at the first and at the second process stage, wherein the first set of data for each process stage comprises measurement results and/or parameters calculated based on measurement results, wherein the measurements and/or parameters are related to raw material, equipment, process measurements and/or product properties;
the measurement elements and the control elements are configured to send the first set of data to the subsequent process stage, and the measurement element and the control element of the subsequent process stage are configured to adjust the process steps of the subsequent process stage based on the first set of data received;
at least the second and the third process stage comprise a measurement element and a control element configured to gather a second set of data at least at the second and at the third process stage, wherein the second set of data for each process stage comprises information related to availability of processing capacity, predicted availability of processing capacity and/or required material flow or quality; and
the measurement elements and the control elements are configured to send the second set of data to the preceding process stage, and the measurement element and the control element of the preceding process stage are configured to wherein the process steps of the preceding process stage are adjusted based on the second set of data received.

9. A control system for chemical pulp production in a multistage process, comprising a control unit configured to cause carrying out the method of claim 1.

10. A computer program product comprising computer-executable code that is configured to, when executed by a computer, to cause carrying out the method of claim 1.

11. A non-transitory memory medium comprising the computer program product of claim 10.
